(19) United States
(12) Patent Application Publication
(10) Pub. No.: US 2006/0042992 A1
(43) Pub. Date: Mar. 2, 2006

(54) Kit and method of utilizing same for promoting sample products

(76) Inventor: Gary A. Vulcano, Scottsdale, AZ (US)

Correspondence Address: Jordan M. Meschkow;Meschkow & Gresham, P.L.C., Suite 409, 5727 North Seventh Street, Phoenix, AZ, 85014, US

(21) Appl. No.: 10/928,365

(22) Filed: Aug. 27, 2004

(51) Int. Cl. A61B 19/02; B65D 69/00

(52) U.S. Cl. 206/581; 206/63.5

(57) ABSTRACT

A sample kit (20) includes a box (28) with a lid (30). A first edge (44) of the lid (30) is in hinged attachment with a top edge(52) of a first wall (23) of the box (28). A second edge (46) of the lid (30) includes a wrap-around tuck flap (54) for connection through a back outer surface (62) of the box (28). A tray (78) is located inside the box (28) and includes compartments (80). Dental or medical practice-related sample products (26) are seated in correspondingly sized ones of the compartments (80). The sample kit (20) further includes a pocket (68) into which literature (72) associated with the sample products (26) is inserted. The sample kit (20) is configured for distribution by a target dental or medical practice.

### TECHNICAL FIELD OF THE INVENTION

The present invention relates to the field of packaging. More specifically, the present invention relates to a kit for providing practice-related sample products from a target practice to clients so as to promote their use.

### BACKGROUND OF THE INVENTION

The prevention of oral disease, such as, dental decay and periodontal disease, is regularly taught to students in the classroom, and to patients in the dentist's office. Indeed, education has long been an important part of preventive and cosemetic dentistry programs. Accompanied by oral explanation, brochures, worksheets, and the like, oral care educators often distribute sample products to their students. These sample products may include a toothbrush, toothpaste, dental floss, mouthwash, plaque disclosing agent, whitening products, and the like.

Unfortunately, the student or patient is typically handed a number of sample products and is expected to stow them in a purse, pocket, or backpack. Alternatively, the student or patient may attempt to carry their handful of sample products as well as associated paperwork, such as, billing information, dental education literature, the dentist's business card, an appointment card listing their next dental visit, and the like.

Attempting to keep all of the information and products together in a organized manner can be inconvenient, and even annoying, for the student or patient. More importantly however, the sample products can get lost, thrown away, and/or damaged under such conditions. Similarly, the associated paperwork can get damaged or separated from the sample products. Consequently, the educational value of the products and literature may become greatly diminished.

Yet another problem with sample product and literature distribution is that the educator is compelled to remember each and every individual component to be provided to the student or patient. Moreover, the educator in a dental office is also compelled to store the assorted boxes, envelopes, and containers of sample products and literature in the dental office, which can take up an undesirable amount of storage space. An educator who visits students has the additional burden of carrying the assorted boxes, envelopes, and containers of sample products and literature to an educational facility, as well as ensuring that he or she has sufficient inventory when visiting the educational facility.

Thus, what is needed is a kit for providing practice-related sample products and associated literature from a target dental or medical practice to their clients.

### SUMMARY OF THE INVENTION

Accordingly, it is an advantage of the present invention that a sample kit and a method for promoting the use of sample products are provided.

It is another advantage of the present invention that a sample kit is provided for a target dental or medical practice that includes practice-related products.

Another advantage of the present invention is that a sample kit is provided for promoting the use of oral care products related to oral hygiene and/or treatment in a convenient, organized, and inexpensive package.

Another advantage of the present invention is that a sample kit is provided that can be used to encourage effective oral care practices within a community by means of promotional distribution and can have provisions for including educational literature and advertising in the kit.

Yet another advantage of the present invention is that a sample kit is provided that is readily stored until its use.

The above and other advantages of the present invention are carried out in one form by a sample kit. The sample kit includes a box and a lid for covering an interior of the box, the lid including a first edge in hinged attachment with a top edge of a first wall of the box and including means, at a second edge, for removable attachment to an outer surface of the box. A tray is located in the interior of the box and includes a plurality of compartments. Sample products are seated in correspondingly sized ones of the plurality of compartments of the tray.

The above and other advantages of the present invention are carried out in another form by a method of promoting the use of sample products by a plurality of clients. The method calls for manufacturing a sample kit for containing the sample products. The manufacturing operation includes creating a tray template defining locations for a number of compartments to be formed in a tray of the sample kit, and fabricating a tray for the sample kit utilizing the tray template, the tray including the compartments. The tray is installed in an interior of a box for the sample kit. The box includes walls surrounding a generally flat bottom, and a lid having peripheral edges corresponding to top edges of the walls of the box. A first one of the peripheral edges is in hinged attachment with one of the top edges of one of said walls, and a second one of the peripheral edges includes means for removable attachment to an outer surface of the box. The manufacturing operation further calls for seating the sample products in correspondingly sized ones of the compartments of the tray, and attaching the lid to the outer surface of the box to enclose the sample products in the box. The method further calls for providing the sample kit to each of the plurality of clients.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 shows a perspective view of a front side of a sample kit in accordance with a preferred embodiment of the present invention;

FIG. 2 shows a perspective view of a rear side of the sample kit of FIG. 1;

FIG. 3 shows a perspective view of the sample kit of FIG. 1 opened to reveal oral hygiene sample products within the sample kit;

FIG. 4 shows a top view of a tray template utilized to fabricate a tray for the sample kit of FIG. 1;

FIG. 5 shows a side sectional view of a tray of the sample kit at a location delineated in FIG. 4 along section lines 5-5; and

FIG. 6 shows a perspective view of a sample kit in accordance with an alternative embodiment of the present invention.

### DETAILED DESCRIPTION OF THE PREFERRED EMBODIMENTS

The present invention provides unique packaging for promoting sample products and their use. In particular, the present invention entails a sample kit for a target dental or medical practice that includes practice-related products. The present invention is described herein as an oral hygiene sample kit for promoting effective oral hygiene practices. The oral hygiene sample kit may be thus utilized by oral hygiene educators to enhance preventive dentistry education in dental offices and in the community. However, the present invention need not be limited to an oral hygiene sample kit or to promoting effective oral hygiene practices, but may instead be adapted for use with a variety of sample products and their associated literature for educational and/or promotional purposes.

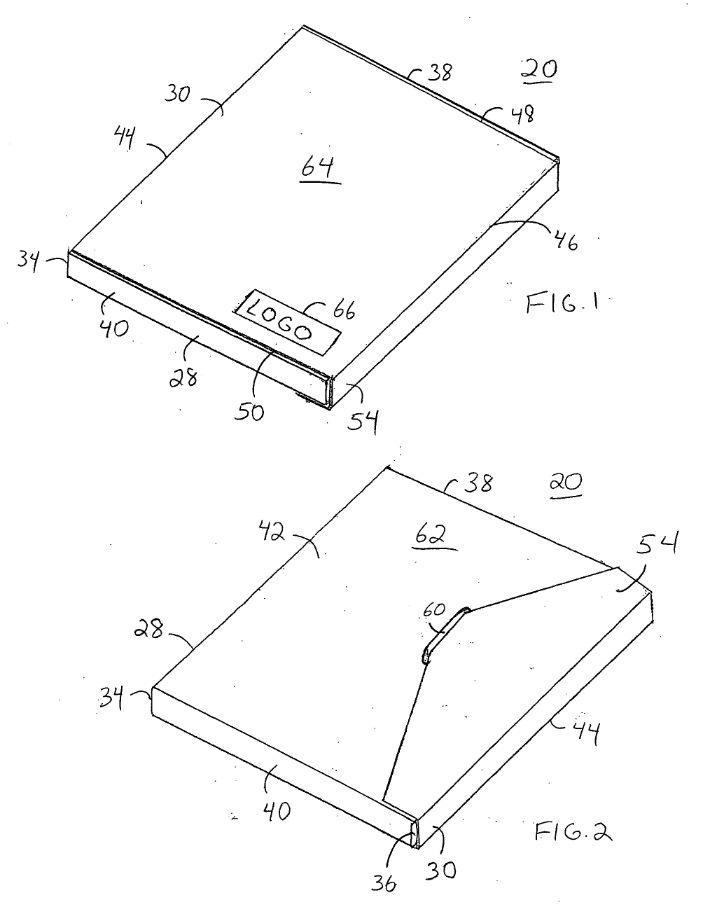

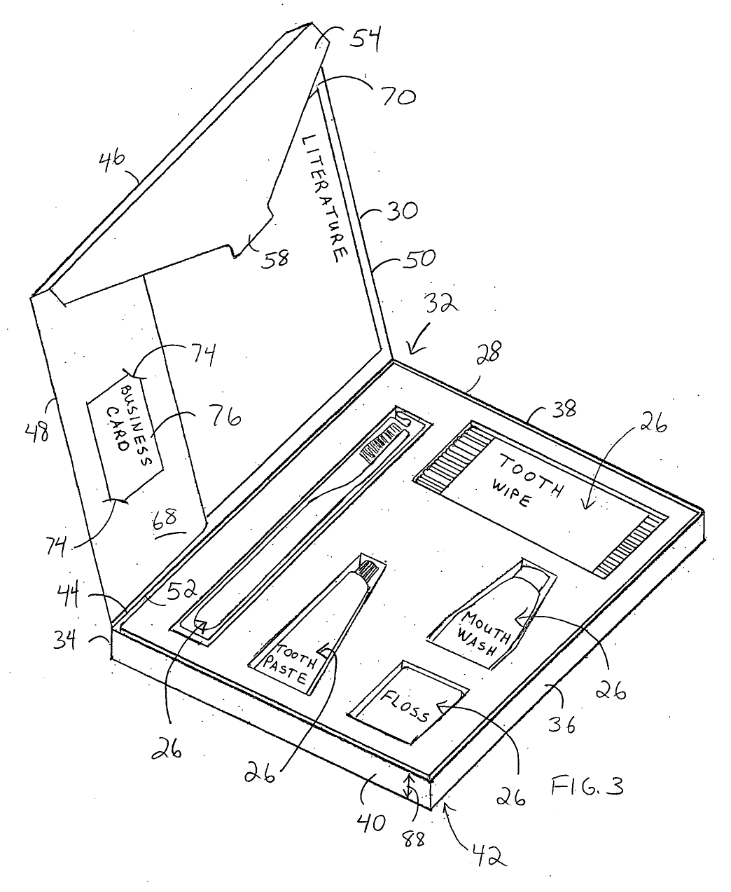

Referring to FIGS. 1-3, FIG. 1 shows a front perspective view of a sample kit 20 in accordance with a preferred embodiment of the present invention, and FIG. 2 shows a rear perspective view of sample kit 20. FIG. 3 shows a perspective view of sample kit 20 opened to reveal product samples 26, in the form of oral hygiene sample products, within kit 22. Oral hygiene sample products 26 may include such things as a toothbrush, toothpaste, dental floss, mouthwash, and a tooth wipe. However, those skilled in the art will recognize that any number and combination of oral hygiene sample products may be included in sample kit 20.

Sample kit 20 includes a box 28 and a lid 30 for covering an interior 32 of box 28. Box 28 includes a first wall 34, a second wall 36, a third wall 38, and a fourth wall 40 surrounding a generally flat bottom 42. Lid 30 is a generally flat member having a first peripheral edge 44, a second peripheral edge 46, a third peripheral edge 48, and a fourth peripheral edge 50. As shown, first, second, third, and fourth peripheral edges 44, 46, 48, and 50 of lid 30 correspond with first, second, third, and fourth walls 34, 36, 38, and 40 of box 28 to enclose the contents of box 28.

First peripheral edge 44 of lid 30 is in hinged attachment with a top edge 52 of first wall 34 of box 28. In an exemplary embodiment, the intersection of first peripheral edge of lid 30 and top edge 52 of first wall 34 of box 28 is a paper hinge. A paper hinge is formed of two pieces of card or cardboard joined on both faces by a cover paper. A small gap is left between the two cards, which then acts as a hinge. A paper hinge technique is employed in sample kit 20 due to its simplicity and low manufacturing cost. However, it should be readily apparent that other hinge means may be employed for allowing lid 30 to be in hinged attachment with first wall 34 of box 28.

Second peripheral edge 46 of lid 30 includes means, in the form of a wrap-around tuck flap 54, for removable attachment to an outer surface of box 28. Wrap-around tuck flap 54 includes a tab 58 sized to be fit into a slot 60 on a back outer surface 62 of box 28. When lid 30 is closed, and tab 58 is secured into slot 60, oral hygiene sample products 26 can be safely and effectively enclosed in box 28. Thus, sample kit 20 presents a low profile, generally rectangular form that can be readily stored. A number of sample kits 20 can be easily stacked and/or bundled into a carton for storage and transport.

In addition to storing oral hygiene sample products 26, sample kit 20 may be used for promotional distribution. As such, a front outer surface 64 of sample kit 20 may include a relevant logo 66. Logo 66 may be the name of the dental office or educational facility distributing sample kit 20. Alternatively, logo 66 may be the manufacturing facility that provides oral hygiene sample products 26.

A pocket 68 is coupled to an inner surface 70 of lid 30. Literature 72 related to sample products 26 may be inserted into pocket 68. Literature 72 may include dental education literature, product information, new patient introductory paperwork provided by the dental office distributing sample kit 20, billing information, and the like. A business card holder, in the form of tabs 74 may be located on pocket 68. A business card 76 of the dental office, business, or marketer distributing sample kit 20 may thus be retained by tabs 74. Although tabs 74 are described as retaining a business card, dental offices often provide their patients with an appointment card that is roughly the size of a business card. This appointment card, providing information regarding their next appointment, may alternatively be retained by tabs 74.

A tray 78 is located in interior 32 of box 28. Tray 78 includes a plurality of compartments 80. As shown, oral hygiene sample products 26 are seated in correspondingly sized ones of compartments 80. In a preferred embodiment, compartments 80 are configured to closely contain sample products 26. That is, each of compartments 80 is sized and shaped to fit a particular item. In addition, each of compartments 80 is spaced apart from other compartments 80. Accordingly, oral hygiene sample products 26 are presented in an organized, aesthetically pleasing, and professional manner that is well suited to promotional distribution.

Tray 78 is removable from sample kit 20. Accordingly, box 28 may be readily adapted for use in connection with a different set of sample products by simply creating a tray that has compartments corresponding to whatever sample products are to be provided. For example, another kit may include a plastic surgery recovery kit that includes post-operative literature and/or instructions and cosmetic products (facial/body lotion, cleanser, concealer makeup, moisturizer, and the like). Such a kit may be distributed by a plastic surgeon associated with a target plastic surgery practice. Similarly, a target eye surgery practice may distribute an eye surgery recovery kit. Such an eye surgery recovery kit might include, for example, post-operative literature and/or instructions, eye drops, medications, sunglasses, movie tickets, and the like.

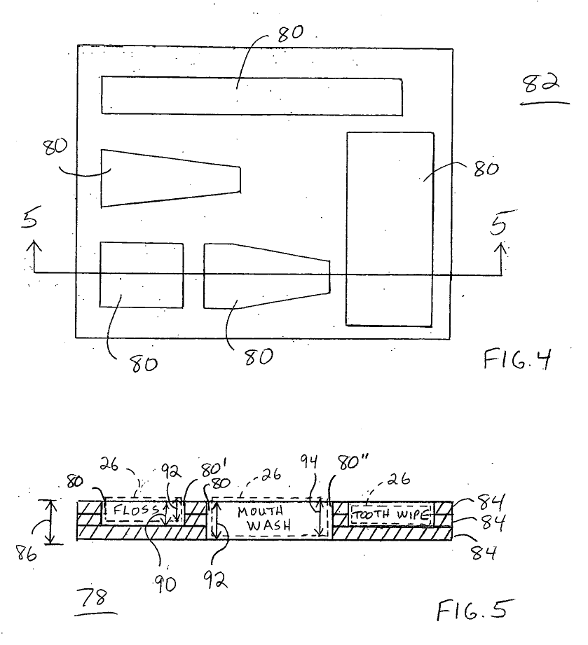

Referring to FIGS. 4-5 in connection with FIG. 3, FIG. 4 shows a top view of a tray template 82 utilized to fabricate tray 78 for sample kit 20, and FIG. 5 shows a side sectional view of tray 78 at a location delineated in FIG. 4 along section lines 5-5.

The manufacture of sample kit 20 (FIG. 3) entails determining the type, quantity, and dimensions of sample products 26 to be included in sample kit 20. Tray template 82 is then created to define locations for compartments 80 as well as to define dimensions for each of compartments 80 corresponding to the unique sizing of each of sample products 26. Tray template 82 may be a paper or cardboard pattern, or may be computer generated.

Once tray template 82 is created, tray 78 can be fabricated. Of course, in an actual manufacturing environment, many trays 78 are fabricated in accordance with tray template 82. In an exemplary embodiment, tray 78 may be a foam core assembly. That is, tray 78 may be formed from at least two layers, and in this case from three layers, of a foam core material 84 that is bonded together. Once foam core material 84 is bonded together, a thickness 86 of tray 78 approximates a depth 88 (see FIG. 3) of box 28. That is, thickness 86 of tray 78 approaches, but desirably does not exceed, depth 88 of box 28.

Foam core material 84 is a stiff sheet of polystyrene foam laminated with paper on both of its sides. It may be any of several thicknesses and is favored for use due to its light weight, stiffness, and ease with which it can be cut. Those skilled in the art, however, will recognize that any of a number of materials may alternatively be employed to fabricate tray 78.

Tray template 82 is utilized to cut out compartments 80 in foam core material 84. As particularly shown in FIG. 5, compartments 80 may be adjusted to varying depths in accordance with the thickness of sample products 26. For example, a depth 90 of a first compartment 80′ corresponds to a thickness 92 of dental floss sample product 26, shown in ghost form, and is approximately two thirds of the total thickness 86 of tray 78. In contrast, a depth 92 of a second compartment 80″ corresponds to a thickness 94 of mouthwash sample product 26, shown in ghost form, and is also equivalent to thickness 86 of tray 78.

Following its fabrication, tray 78 is installed into interior 32 of box 28 and oral hygiene sample products are seated in their correspondingly size compartments 80. Literature 72 related to sample products 26 is inserted in pocket 68, and business card 76 is placed in business card holder, i.e., retained by tabs 74. Lid 30 is closed and tab 58 of tuck flap 54 is inserted in slot 60 (FIG. 2) in back outer surface 62 (FIG. 2) of box 28. When tab 58 is attached to box 28 via slot 60 in back outer surface 62, lid 30 effectively encloses sample products 26.

A method of promoting the use of sample products 26 by a plurality of clients entails manufacturing sample kit 20 as discussed above, and subsequently providing sample kit 20 to each of a plurality of clients, i.e., students, patients, and the like. Of course, pursuant to conventional practices, following the manufacture of sample kit 20, a number of sample kits 20 may be packed into cartons, and distributed to dental offices, schools, and the like for subsequent provision to the plurality of clients.

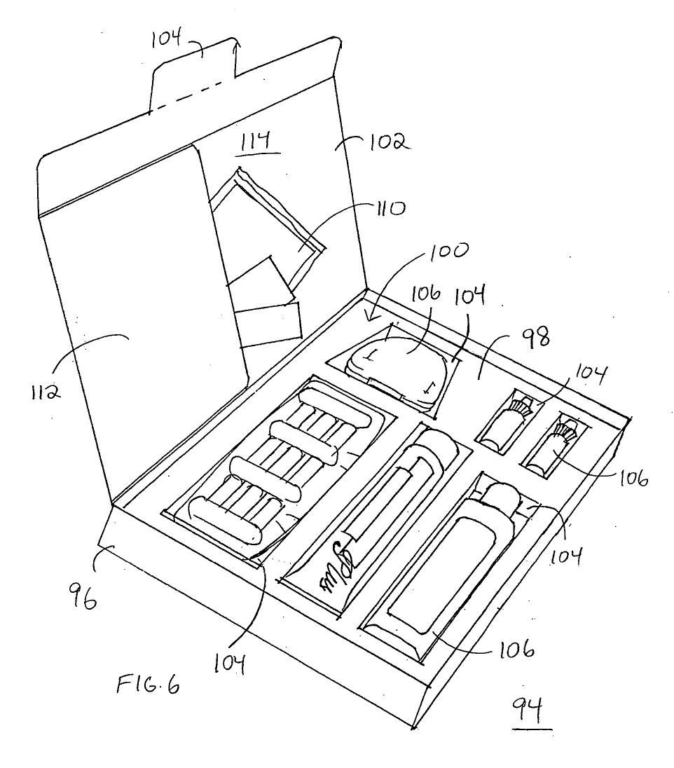

FIG. 6 shows a perspective view of a sample kit 94 in accordance with an alternative embodiment of the present invention. Sample kit 94 is similar to sample kit 20 in that it includes a box 96 and a tray 98 seated in an interior 100 of box 96. A lid 102 is in hinged attachment with box 96, and further includes a flap 104 for removable attachment to box 96.

Tray 98 includes a plurality of compartments 104. As shown, oral treatment products 106 are seated in correspondingly sized ones of compartments 108. In a preferred embodiment, compartments 108 are configured to closely contain oral treatment products 106, and each of compartments 108 is sized and shaped to fit a particular item. In addition, each of compartments 108 is spaced apart from other compartments 108. In a preferred embodiment, oral treatment products 106 are related to a tooth whitening process. Accordingly, oral treatment products 106 may include tooth whitening toothpaste, whitening rinse, whitening gel, a mouthguard and case, breath drops, and the like. In addition, literature 110 related to tooth whitening is included in a pocket 112 located on an inner surface 114 of box 96. Accordingly, oral treatment sample products 106 are presented in an organized, aesthetically pleasing, and professional manner that is well suited to promotional distribution.

In summary, the present invention teaches of a sample kit and a method for promoting the use of sample products. The sample kit includes a tray that retains the sample products in an aesthetically pleasing arrangement. The sample kit facilitates the promotional distribution and use of oral hygiene and/or treatment sample products in a convenient, organized, and inexpensive package, thus encouraging effective oral hygiene and treatment practices within a community. The sample kit also includes a pocket and a business card holder for the inclusion of educational literature and advertising in the kit. The low profile and generally rectangular shape of sample kit facilitate its ready storage and transport until its use. Moreover, since the sample products, literature, and advertising are included in a single package, inventory control is simplified and an educator can effectively distribute all of the desired materials.

Although the preferred embodiments of the invention have been illustrated and described in detail, it will be readily apparent to those skilled in the art that various modifications may be made therein without departing from the spirit of the invention or from the scope of the appended claims. For example, the configuration of the tray is readily modified to include different oral hygiene sample products, or to include sample products unrelated to oral hygiene.

What is claimed is:

1. A sample kit comprising:
a box;
a lid for covering an interior of said box, said lid including a first edge in hinged attachment with a top edge of a first wall of said box and including means, at a second edge, for removable attachment to an outer surface of said box;
a tray located in said interior of said box, said tray including a plurality of compartments; and
sample products seated in correspondingly sized ones of said plurality of compartments of said tray.

2. A sample kit as claimed in claim 1 wherein:
said box includes walls surrounding a generally flat bottom, said first wall being one of said walls; and
said lid is a generally flat member having peripheral edges corresponding to top edges of said walls of said box, said first and second edges being ones of said peripheral edges.

3. A sample kit as claimed in claim 1 wherein said removable attachment means includes a wrap-around tuck flap for connection through a back outer surface of said box.

4. A sample kit as claimed in claim 1 wherein depths of said correspondingly sized ones of said plurality of compartments in said tray correspond to thicknesses of said sample products.

5. A sample kit as claimed in claim 1 wherein each of said plurality of compartments is spaced apart from others of said plurality of compartments in said tray.

6. A sample kit as claimed in claim 1 wherein said tray is a foam core assembly exhibiting a thickness approximating a depth of said box.

7. A sample kit as claimed in claim 6 wherein said foam core assembly includes at least two layers of a foam core material bonded together.

8. A sample kit as claimed in claim 1 wherein said correspondingly sized ones of said plurality of compartments are configured to closely contain said sample products.

9. A sample kit as claimed in claim 1 further comprising:
a pocket coupled to an inner surface of said lid; and
literature related to said sample products inserted into said pocket.

10. A sample kit as claimed in claim 1 further comprising:
a business card holder associated with an inner surface of said lid; and
a business card retained by said business card holder.

11. A sample kit as claimed in claim 1 wherein said sample products include dental practice-related products.

12. A sample kit as claimed in claim 1 wherein said sample products include medical practice-related products.

13. A method of promoting the use of sample products by a plurality of clients, said method comprising:
manufacturing a sample kit for containing said sample products, said manufacturing operation including:
creating a tray template defining locations for a number of compartments to be formed in a tray of said sample kit;
fabricating a tray for said sample kit utilizing said tray template, said tray including said compartments;
installing said tray in an interior of a box for said sample kit, said box including walls surrounding a generally flat bottom, and a lid having peripheral edges corresponding to top edges of said walls of said box, wherein a first one of said peripheral edges is in hinged attachment with one of said top edges of one of said walls, and a second one of said peripheral edges includes means for removable attachment to an outer surface of said box;
seating said sample products in correspondingly sized ones of said compartments of said tray;
attaching said lid to said outer surface of said box to enclose said sample products in said box; and
providing said sample kit to each of said plurality of clients.

14. A method as claimed in claim 13 wherein said manufacturing operation further includes adjusting depths of said correspondingly sized ones of said compartments to correspond to thicknesses of said sample products.

15. A method as claimed in claim 13 wherein said creating operation includes positioning each of said compartments in spaced-apart relation from others of said plurality of compartments in said tray template.

16. A method as claimed in claim 13 wherein said fabricating operation comprises bonding at least two layers of a foam core material together to yield a foam core assembly exhibiting a thickness approximating a depth of said box.

17. A method as claimed in claim 13 further comprising inserting literature related to said sample products in a pocket coupled to said box.

18. A sample kit for a target dental or medical practice comprising:
a box;
a lid for covering an interior of said box, said lid including a first edge in hinged attachment with a top edge of a first wall of said box and including means, at a second edge, for removable attachment to an outer surface of said box;
a pocket coupled to an inner surface of said lid;
a tray located in said interior of said box, said tray including a plurality of compartments;
practice-related products seated in correspondingly sized ones of said plurality of compartments of said tray; and
literature related to said practice-related products inserted into said pocket.

19. A sample kit as claimed in claim 18 wherein said removable attachment means includes a wrap-around tuck flap for connection to a back surface of said box.

20. A sample kit as claimed in claim 18 further comprising:
a business card holder formed on said pocket; and
a business card retained by said business card holder.
